(19) United States
(12) Patent Application Publication
(10) Pub. No.: US 2026/0367094 A1
(43) Pub. Date: Nov. 19, 2026

(54) IDENTIFICATION OF CONTACT LENS WEARERS PREDISPOSED TO CONTACT LENS DISCOMFORT

(71) Applicant: the University of Melbourne, Victoria (AU)

(72) Inventors: Laura Elizabeth DOWNIE, Victoria (AU); Chinn Yi WONG, Victoria (AU); David Charles JACKSON, Victoria (AU)

(21) Appl. No.: 19/138,576

(22) PCT Filed: Dec. 15, 2023
(86) PCT No.: PCT/AU2023/051310

§ 371 Date: Jun. 12, 2025

(30) Foreign Application Priority Data
Dec. 16, 2022 (AU) 2022903870

(51) Int. Cl. G01N 33/68

(52) U.S. Cl. CPC G01N 33/6869; G01N 2333/54; G01N 2800/52

(57) ABSTRACT

The present disclosure relates to methods for identifying contact lens wearers predisposed to contact lens discomfort based on basal tear level of one or more of interleukins.

### TECHNICAL FIELD

The field of the invention relates to methods for identifying contact lens wearers predisposed to contact lens discomfort.

### BACKGROUND OF INVENTION

Inflammation is a complex non-specific tissue response elicited by exposure to potentially harmful stimuli, and is a feature of several ocular surface conditions, including dry eye disease, conjunctivochalasis, ocular allergy and blepharitis. In these clinical presentations, the anterior eye response typically features classic signs of inflammation, including redness, pain, swelling, heat and loss of normal function. These clinical signs are accompanied by alterations to the expression of inflammatory biomarkers, such as human leukocyte antigen (HLA-DR) and intercellular adhesion molecule-1 (ICAM-1), on ocular surface cells. Furthermore, there are changes to a tear film composition, including an upregulation of pro-inflammatory cytokines, alterations to tear lipid mediators and increased tear protease activity (e.g., metalloproteinases, MMP-9 and MMP-2).

Although uncomplicated contact lens wear is not associated with these classic signs of inflammation, the contribution of inflammation to the discomfort response experienced by some contact lens wearers remains controversial. Contact lens discomfort is a multi-factorial condition characterized by episodic or persistent adverse ocular sensations related to contact lens wear that results in discontinuation of contact lenses. At present, the etiology of this discomfort response is unclear. Notably, symptoms reported by patients experiencing symptomatic lens wear (e.g., eye irritation, burning, stinging and pain) are highly similar to the symptomatology associated with dry eye disease, which is recognized to involve an inflammatory overlay.

Some clinical data lend support to the presence of a low grade, sub-clinical anterior eye inflammatory response during contact lens wear that may be exaggerated in people who experience discomfort. Contact lens wear alters the relative levels of a range of tear film mediators. However, in contrast to dry eye disease where levels of tear pro-inflammatory cytokines are elevated, and also implicated in the disease process, a link between ocular discomfort and the upregulation of specific inflammatory mediators is less clear. Changes to key tear film mediators, including the complement cascade, kinin cascade and histamine, do not appear to be associated with comfort levels. Roles for neurokine growth factor, which can induce hyperalgesia and mast cell degranulation, and the prolactin-induced protein, which modulates water transport in the lacrimal gland, have been proposed. Other potential molecular candidates include degraded lipids, leukotriene-B4 and peroxidation products, and the enzyme, secretory phospholipase-A2.

It would be desirable to identify contact lens wearers or prospective contact lens wearers who may be predisposed to developing contact lens discomfort so that proactive steps may be taken to improve the contact lens-wearing experience of these patients.

### SUMMARY OF INVENTION

An immunological and clinical characterization of the ocular inflammatory response in symptomatic soft contact lens wearers compared to asymptomatic wearers was undertaken to gain insight into the nature of the inflammatory response occurring in people experiencing contact lens discomfort.

The present inventors have surprisingly found that individuals predisposed to symptomatic contact lens wear can be distinguished based on their basal tear level of one or more of interleukin-23 (IL-23), interleukin-33 (IL-33), and interleukin-18 (IL-18). Accordingly, the present methods allow for the identification of individual predisposed to symptomatic contact lens wear and treatment thereof to prevent, inhibit, or reduce discomfort associated with contact lens wear.

Accordingly, the present disclosure provides a method of determining a predisposition to symptomatic contact lens wear in a patient said method comprising:
a) determining the concentration of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33,

wherein a higher level of the one or more of IL-18, IL-23, and IL-33 in the tear sample compared to the reference level is indicative of a predisposition of the patient to symptomatic contact lens wear.

The present disclosure also provides a method of determining a predisposition to symptomatic contact lens wear in a patient said method comprising:
a) determining the concentration of one or both of interleukin-18 (IL-18), and interleukin-33 (IL-33) in a tear sample from the patient; and
b) comparing the level of IL-18 or IL-33, or IL-18 and IL-33 in the tear sample to a reference level of IL-18 or IL-33, or IL-18 and IL-33, respectively,

wherein a higher level of one or both of IL-18 and IL-33 in the tear sample compared to the reference level is indicative of a predisposition of the patient to symptomatic contact lens wear.

In one embodiment, the method further comprises determining the concentration of interleukin-23 (IL-23) in the tear sample and comparing the level of IL-23 in the tear sample to a reference level of IL-23, wherein a higher level of IL-23 in the tear sample compared to the reference level is indicative of a predisposition of the patient to symptomatic contact lens wear.

In one embodiment, the level of the one or more of IL-18, IL-23, and IL-33 in the tear sample is determined prior to contact lens wear.

In one or a further embodiment, the reference level of the one or more of IL-18, IL-23, and IL-33 is a level of the one or more of IL-18, IL-23, and IL-33 in a sample taken prior to contact lens wear from a subject who is asymptomatic to contact lens wear.

In some embodiments, the reference level of the one or more of IL-18, IL-23, and IL-33 is a threshold level.

In one embodiment, the threshold level is a cut-off value.

In some embodiments, the method further comprises obtaining the tear sample from the patient.

In some embodiments, the level of the one or more of one of IL-18, IL-23, and IL-33 is determined using an immunoassay such as a multiplex cytometric bead array.

In one embodiment, the threshold level for:
i) tear IL-18 is about >128 pg/ml; or
ii) tear IL-23 is between about >29 to about >46 pg/ml; or
iii) tear IL-33 is between about >170 to >368 pg/ml.

In another embodiment, the threshold level for:
i) tear IL-23 is about >50 pg/ml OR tear IL-33 is about >200 pg/ml; or
ii) tear IL-23 about >50 pg/ml OR tear IL-33 is about >370 pg/ml; or
iii) tear IL-23 is about >50 pg/ml OR tear IL-33 is about >370 pg/ml OR tear IL-18 is about >128 pg/ml.

In some embodiments, the method further comprises applying a scaling factor to the level of one or more of one of IL-18, IL-23, and IL-33.

In one embodiment, the threshold level is a weighted threshold level calculated using the formula:
i) (tear IL-23×scaling factor A)+(tear IL-33×scaling factor B); or
ii) (tear IL-23×scaling factor A)+(tear IL-18×scaling factor C); or
iii) (tear IL-33×scaling factor B)+(tear IL-18×scaling factor C); or
iv) (tear IL-23×scaling factor A)+(tear IL-33×scaling factor B)+(tear IL-18×scaling factor C),
   wherein scaling factor A, B and C are independently selected, to for example, improve sensitivity or specificity or both sensitivity and specificity of the method.

In one embodiment, the weighted threshold level is:

(tear IL-23×about 2)+(tear IL-33×about 0.5))>about 160 pg/ml.

In some embodiments, the method further comprises selecting a treatment or modifying a treatment, based on the diagnosis of a predisposition of the patient to symptomatic contact lens wear.

In one embodiment, the treatment is for preventing, inhibiting, or reducing discomfort associated with contact lens wear and is selected from administration of a therapeutic agent for neuropathy or ocular pain or discomfort, an anti-inflammatory agent, a wetting agent, an immunomodulatory agent, a tear composition modifying agent, a viscoelastic agent, a different contact lens, and combinations thereof.

In some embodiments, the treatment is given prophylactically to prevent or reduce discomfort associated with contact lens wear.

In other or further embodiments, the treatment is given during contact lens wear to inhibit or reduce severity of discomfort associated with contact lens wear.

In another embodiment, the treatment is for preventing or reducing discomfort associated with contact lens wear and comprises prescribing the patient spectacle lenses for vision correction.

In some embodiments, the patient does not have Sjogren's syndrome or dry eye disease.

The present disclosure also provides a method for selecting a treatment for a patient in need of vision correction, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of L-18, IL-23, and IL-33; and
c) selecting a treatment for preventing, inhibiting, or reducing discomfort associated with contact lens wear when the level of the one or more of IL-18, IL-23, and IL-33 is higher in the tear sample compared to the reference level.

In one embodiment, the level of the one or more of IL-18, IL-23, and IL-33 in the tear sample is determined prior to contact lens wear.

In one or a further embodiment, the reference level of the one or more of IL-18, IL-23, and IL-33 is a level of the one or more of IL-18, IL-23, and IL-33 in a sample taken prior to contact lens wear from a subject who is asymptomatic to contact lens wear.

In some embodiments, the reference level of the one or more of IL-18, IL-23, and IL-33 is a threshold level.

In one embodiment, the threshold level is a cut-off value.

In some embodiments, the method further comprises obtaining the tear sample from the patient.

In some embodiments, the level of the one or more of one of IL-18, IL-23, and IL-33 is determined using an immunoassay such as a multiplex cytometric bead array.

In one embodiment, the threshold level for:
i) tear IL-18 is about >128 pg/ml; or
ii) tear IL-23 is between about >29 to about >46 pg/ml; or
iii) tear IL-33 is between about >170 to >368 pg/ml.

In another embodiment, the threshold level for:
i) tear IL-23 is about >50 pg/ml OR tear IL-33 is about >200 pg/ml; or
ii) tear IL-23 about >50 pg/ml OR tear IL-33 is about >370 pg/ml; or
iii) tear IL-23 is about >50 pg/ml OR tear IL-33 is about >370 pg/ml OR tear IL-18 is about >128 pg/ml.

In some embodiments, the method further comprises applying a scaling factor to the level of one or more of one of IL-18, IL-23, and IL-33.

In one embodiment, the threshold level is a weighted threshold level calculated using the formula:
i) (tear IL-23×scaling factor A)+(tear IL-33×scaling factor B);
ii) (tear IL-23×scaling factor A)+(tear IL-18×scaling factor C); or
iii) (tear IL-33×scaling factor B)+(tear IL-18×scaling factor C); or
iv) (tear IL-23×scaling factor A)+(tear IL-33×scaling factor B)+(tear IL-18×scaling factor C),
   wherein scaling factor A, B and C are independently selected, to for example, improve sensitivity or specificity or both sensitivity and specificity of the method.

In one embodiment, the weighted threshold level is:

(tear IL-23×about 2)+(tear IL-33×about 0.5))>about 160 pg/ml.

In one embodiment, the treatment is for preventing, inhibiting, or reducing discomfort associated with contact lens wear and is selected from administration of a therapeutic agent for neuropathy or ocular pain or discomfort, an anti-inflammatory agent, a wetting agent, an immunomodulatory agent, a tear composition modifying agent, a viscoelastic agent, a different contact lens, and combinations thereof.

In some embodiments, the treatment is given prophylactically to prevent or reduce discomfort associated with contact lens wear.

In other or further embodiments, the treatment is given during contact lens wear to inhibit or reduce severity of discomfort associated with contact lens wear.

In another embodiment, the treatment is for preventing or reducing discomfort associated with contact lens wear and comprises prescribing the patient spectacle lenses for vision correction.

In some embodiments, the patient does not have Sjogren's syndrome or dry eye disease.

The present disclosure also provides a method for treating a patient in need of vision correction, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33; and
c) prescribing the patient a treatment for preventing, inhibiting, or reducing discomfort associated with contact lens wear when the level of the one or more of IL-18, IL-23, and IL-33 is higher in the tear sample compared to the reference level.

The present disclosure also provides a method for treating a patient in need of vision correction, said method comprising:
a) determining the level of one or both of interleukin-18 (IL-18), and interleukin-33 (IL-33) in a tear sample from the patient; and
b) comparing the level of IL-18 or IL-33, or IL-18 and IL-33 in the tear sample to a reference level of IL-18 or IL-33, or IL-18 and IL-33, respectively; and
c) prescribing the patient a treatment for preventing, inhibiting, or reducing discomfort associated with contact lens wear when the level of one or both of IL-18 and IL-33 is higher in the tear sample compared to the reference level.

In one embodiment, the method further comprises determining the concentration of interleukin-23 (IL-23) in the tear sample and comparing the level of IL-23 in the tear sample to a reference level of IL-23; and prescribing the patient a treatment for preventing, inhibiting, or reducing discomfort associated with contact lens wear when the level of IL-23 is higher in the tear sample compared to the reference level.

In one embodiment, the level of the one or more of IL-18, IL-23, and IL-33 in the tear sample is determined prior to contact lens wear.

In one or a further embodiment, the reference level of the one or more of IL-18, IL-23, and IL-33 is a level of the one or more of IL-18, IL-23, and IL-33 in a sample taken prior to contact lens wear from a subject who is asymptomatic to contact lens wear.

In some embodiments, the reference level of the one or more of IL-18, IL-23, and IL-33 is a threshold level.

In one embodiment, the threshold level is a cut-off value.

In some embodiments, the method further comprises obtaining the tear sample from the patient.

In some embodiments, the level of the one or more of one of IL-18, IL-23, and IL-33 is determined using an immunoassay such as a multiplex cytometric bead array.

In one embodiment, the threshold level for:
i) tear IL-18 is about >128 pg/ml; or
ii) tear IL-23 is between about >29 to about >46 pg/ml; or
iii) tear IL-33 is between about >170 to >368 pg/ml.

In another embodiment, the threshold level for:
i) tear IL-23 is about >50 pg/ml OR tear IL-33 is about >200 pg/ml; or
ii) tear IL-23 about >50 pg/ml OR tear IL-33 is about >370 pg/ml; or
iii) tear IL-23 is about >50 pg/ml OR tear IL-33 is about >370 pg/ml OR tear IL-18 is about >128 pg/ml.

In some embodiments, the method further comprises applying a scaling factor to the level of one or more of one of IL-18, IL-23, and IL-33.

In one embodiment, the threshold level is a weighted threshold level calculated using the formula:
i) (tear IL-23×scaling factor A)+(tear IL-33×scaling factor B); or
ii) (tear IL-23×scaling factor A)+(tear IL-18×scaling factor C); or
iii) (tear IL-33×scaling factor B)+(tear IL-18×scaling factor C); or
iv) (tear IL-23×scaling factor A)+(tear IL-33×scaling factor B)+(tear IL-18×scaling factor C),
   wherein scaling factor A, B and C are independently selected, to for example, improve sensitivity or specificity or both sensitivity and specificity of the method.

In one embodiment, the weighted threshold level is:

(tear IL-23×about 2)+(tear IL-33×about 0.5))>about 160 pg/ml.

In one embodiment, the treatment is for preventing, inhibiting, or reducing discomfort associated with contact lens wear and is selected from administration of a therapeutic agent for neuropathy or ocular pain or discomfort, an anti-inflammatory agent, a wetting agent, an immunomodulatory agent, a tear composition modifying agent, a viscoelastic agent, a different contact lens, and combinations thereof.

In some embodiments, the treatment is given prophylactically to prevent or reduce discomfort associated with contact lens wear.

In other or further embodiments, the treatment is given during contact lens wear to inhibit or reduce severity of discomfort associated with contact lens wear.

In another embodiment, the treatment is for preventing or reducing discomfort associated with contact lens wear and comprises prescribing the patient spectacle lenses for vision correction.

In some embodiments, the patient does not have Sjogren's syndrome or dry eye disease.

The present disclosure also provides a method of monitoring treatment efficacy of a patient in need of vision correction and having a predisposition to symptomatic contact lens, or a patient in need of vision correction having symptomatic contact lens wear, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33; and
c) optionally, continuing treatment when the subject is determined to be a responder or has a decreased level of the one or more of IL-18, IL-23, and IL-33 compared to the reference level; or switching the treatment when the subject is determined to be a non-responder, wherein switching the treatment comprises increasing a dose of the treatment administered, adding one or more of agents to the treatment regimen, or discontinuing the treatment and initiating a different treatment.

The present disclosure also provides a method of monitoring treatment efficacy of a patient in need of vision correction and having a predisposition to symptomatic contact lens, or a patient in need of vision correction having symptomatic contact lens wear, said method comprising:
a) determining the level of one or both of interleukin-18 (IL-18) and interleukin-33 (IL-33) in a tear sample from the patient; and
b) comparing the level of IL-18 or IL-33, or IL-18 and IL-33 in the tear sample to a reference level of IL-18 or IL-33, or IL-18 and IL-33, respectively; and
c) optionally, continuing treatment when the subject is determined to be a responder or has a decreased level of one or both of IL-18 and IL-33 compared to the reference level; or switching the treatment when the subject is determined to be a non-responder, wherein switching the treatment comprises increasing a dose of the treatment administered, adding one or more of agents to the treatment regimen, or discontinuing the treatment and initiating a different treatment.

In one embodiment, the method further comprises determining the concentration of interleukin-23 (IL-23) in the tear sample and comparing the level of IL-23 in the tear sample to a reference level of IL-23; and optionally, continuing treatment when the subject is determined to be a responder or has a decreased level of IL-23 compared to the reference level; or switching the treatment when the subject is determined to be a non-responder, wherein switching the treatment comprises increasing a dose of the treatment administered, adding one or more of agents to the treatment regimen, or discontinuing the treatment and initiating a different treatment.

In one embodiment, the level of the one or more of IL-18, IL-23, and IL-33 in the tear sample is determined prior to contact lens wear.

In one or a further embodiment, the reference level of the one or more of IL-18, IL-23, and IL-33 is a level of the one or more of IL-18, IL-23, and IL-33 in a sample taken from a subject prior to contact lens wear from a subject who is asymptomatic to contact lens wear.

In some embodiments, the reference level of the one or more of IL-18, IL-23, and IL-33 is a threshold level.

In one embodiment, the threshold level is a cut-off value.

In some embodiments, the method further comprises obtaining the tear sample from the patient.

In some embodiments, the level of the one or more of one of IL-18, IL-23, and IL-33 is determined using an immunoassay such as a multiplex cytometric bead array.

In one embodiment, the threshold level for:
i) tear IL-18 is about >128 pg/ml; or
ii) tear IL-23 is between about >29 to about >46 pg/ml; or
iii) tear IL-33 is between about >170 to >368 pg/ml.

In another embodiment, the threshold level for:
i) tear IL-23 is about >50 pg/ml OR tear IL-33 is about >200 pg/ml; or
ii) tear IL-23 about >50 pg/ml OR tear IL-33 is about >370 pg/ml; or
iii) tear IL-23 is about >50 pg/ml OR tear IL-33 is about >370 pg/ml OR tear IL-18 is about >128 pg/ml.

In some embodiments, the method further comprises applying a scaling factor to the level of one or more of one of IL-18, IL-23, and IL-33.

In one embodiment, the threshold level is a weighted threshold level calculated using the formula:
i) (tear IL-23×scaling factor A)+(tear IL-33×scaling factor B); or
ii) (tear IL-23×scaling factor A)+(tear IL-18×scaling factor C); or
iii) (tear IL-33×scaling factor B)+(tear IL-18×scaling factor C); or
iv) (tear IL-23×scaling factor A)+(tear IL-33×scaling factor B)+(tear IL-18×scaling factor C),
   wherein scaling factor A, B and C are independently selected, to for example, improve sensitivity or specificity or both sensitivity and specificity of the method.

In one embodiment, the weighted threshold level is:

(tear IL-23×about 2)+(tear IL-33×about 0.5))>about 160 pg/ml.

In one embodiment, the treatment is for preventing, inhibiting, or reducing discomfort associated with contact lens wear and is selected from administration of a therapeutic agent for neuropathy or ocular pain or discomfort, an anti-inflammatory agent, a wetting agent, an immunomodulatory agent, a tear composition modifying agent, a viscoelastic agent, a different contact lens, and combinations thereof.

In some embodiments, the treatment is given prophylactically to prevent or reduce discomfort associated with contact lens wear.

In other or further embodiments, the treatment is given during contact lens wear to inhibit or reduce severity of discomfort associated with contact lens wear.

In another embodiment, the treatment is for preventing or reducing discomfort associated with contact lens wear and comprises prescribing the patient spectacle lenses for vision correction.

In some embodiments, the patient does not have Sjogren's syndrome or dry eye disease.

The present disclosure also provides the use of one or more of interleukin-18 (IL-18), interleukin-23 (IL-23), and interleukin-33 (IL-33) as a tear biomarker for predisposition to symptomatic contact lens wear.

The present disclosure also provides the use of one or both of interleukin-18 (IL-18) and interleukin-33 (IL-33) as a tear biomarker for predisposition to symptomatic contact lens wear.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### FIGURES

FIG. 1: Overall ocular (eye) comfort (/100), scored on a visual analogue scale (VAS) from 0 to 100 (where higher scores indicate better eye comfort), for asymptomatic contact lens (CL) wearers (n=11) and symptomatic CL wearers (n=16) at each study visit. The study visits comprised a baseline visit (Visit 1 before CL wear), and then three sub-visits on the same day, approximately one week after Visit 1 (baseline): Visit 2 prior to CL wear, after 3.5 hours of CL wear (Visit 3) and after 7 hours of CL wear (Visit 4). Individuals with CL discomfort show significantly lower ocular comfort scores at the 7 hour time-point. Data shown as mean±SEM of the right and left eyes. * indicates p<0.05 between groups.

FIG. 2: Tear CGRP levels (ng/ml) at each study visit for the study population and the study visits as described for FIG. 1. There are no significant differences in tear levels of this neuropeptide between study groups (i.e., asymptomatic and symptomatic CL wearers), at any of the study visits (p>0.05 for all comparisons).

FIG. 3: Tear IL-23 and IL-33 (pg/ml) levels at each study visit, for asymptomatic contact lens (CL) wearers (n=11) and symptomatic CL wearers (n=16) at each study visit. The study visits comprised a baseline visit (Visit 1 before CL wear), and then three sub-visits on the same day, approximately one week after Visit 1 (baseline): Visit 2 prior to CL wear, after 3.5 hours of CL wear (Visit 3) and after 7 hours of CL wear (Visit 4). Individuals who developed CL discomfort showed significantly higher tear IL-23 (panel A) and tear IL-33 levels (panel B) at baseline (prior to CL wear). There was a strong linear relationship (R²=0.69, p<0.0001) between tear IL-23 and IL-33 levels at Visit 2 (panel C). Data shown as mean±SEM of the right and left eyes. * indicates p<0.05 between groups.

FIG. 4: Tear IL-18 (panel A) and IL-8 levels (panel B) (pg/ml) each study visit, for asymptomatic contact lens (CL) wearers (n=11) and symptomatic CL wearers (n=16) at each study visit. The study visits comprised prior to CL wear (Visit 1, with a 24-hour washout period with no CL wear), prior to CL wear on the day CLs were to be worn (Visit 2), after 3.5 hours of CL wear (Visit 3) and after 7 hours of CL wear (Visit 4). Individuals who developed CL discomfort showed a trend towards higher tear IL-18 levels (panel A) at baseline (prior to CL wear). There were no significant inter-group differences at any study visit for tear IL-8 levels. Data shown as mean±SEM of the right and left eyes.

FIG. 5: Receiver operator characteristic (ROC) curve analysis for tear IL-23 levels at Visit 2 (prior to CL wear), to investigate prognostic utility to identify individuals predisposed to CL discomfort. Area Under the Curve (AUC): 0.81; standard error: 0.085; 95% CI: 0.65 to 0.98 (p=0.0075). Sensitivity/specificity table shown below the ROC curve.

FIG. 6: Receiver operator characteristic (ROC) curve analysis for tear IL-33 levels at Visit 2 (prior to CL wear), to investigate prognostic utility to identify individuals predisposed to CL discomfort. Area Under the Curve (AUC): 0.70; standard error: 0.10; 95% CI: 0.50 to 0.91 (p=0.08). Sensitivity/specificity table shown below the ROC curve.

FIG. 7: Receiver operator characteristic (ROC) curve analysis for tear IL-18 levels at Visit 2 (prior to CL wear), to investigate prognostic utility to identify individuals predisposed to CL discomfort. Area Under the Curve (AUC): 0.65; standard error: 0.11; 95% CI: 0.43 to 0.87 (p=0.20). Sensitivity/specificity table shown below the ROC curve.

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art

All publications or patent applications or patents cited herein are entirely incorporated herein by reference.

A reference herein to a publication or patent application or patent or other matter which is given as prior art is not to be taken as admission that the document or matter (e.g., databases) was known or that the information it contains was part of the common general knowledge as at the priority date of any of the claims.

The present disclosure relates to methods for determining a pre-disposition to symptomatic contact lens wear in a patient in need of vision correction, as well as methods of treatment for preventing, inhibiting or reducing discomfort associated with contact lens wear. The methods comprise determining the level of one or more of IL-18, IL-23, and IL-33 in a tear fluid sample from the patient and comparing the level of the one or more of IL-18, IL-23, and IL-33 to a reference level of the one or more of IL-18, IL-23, and IL-33.

As used herein the term “predisposition” is defined as a tendency or susceptibility to manifest a condition (i.e., symptomatic contact lens wear). A patient is more likely than a control subject to manifest the condition.

Successful contact lens wear has been defined as being able to comfortably wear one's contact lenses for at least 12 hours per day for at least six days per week while still being able to see at least as well as while wearing spectacles.

“Symptomatic contact lens wear” is generally characterized by the lens wearer experiencing discomfort when the lens (lenses) has (have) been worn for 3 or more continuous hours. A patient with symptomatic contact lens wear may experience one or more of the following symptoms burning, itching, or stinging of the eye(s), sensation of something being stuck in the eye(s), excessive watering or tearing of the eye(s), unusual eye(s) secretion(s), redness of the eye(s), reduced sharpness of vision, blurred vision, rainbows, or halos around objects, and sensitivity to light.

As used herein, “contact lens” can refer to a soft or hard contact lens or a hybrid contact lens. Soft contact lenses may be daily disposable or planned replacement. Contact lenses may be for daily wear or extended wear.

“Soft contact lenses” are made of soft, flexible plastics (e.g., hydrogel or silicon hydrogel polymers that allow oxygen to pass through to the cornea. This lens material may be easier to adjust to and provide better initial comfort than hard, or rigid gas permeable, contact lenses. Soft contact lenses are the most common lens material worn.

“Hard, or rigid gas permeable (RGP) contact lenses” are generally more durable than soft contact lenses and are resistant to build-up of eye-produced deposits on the lens surface. Hard contact lenses generally provide clearer, crisper vision. They also tend to be less expensive over the life of the lens since they last longer than soft contact lenses. Hard contact lenses are easier to handle and less likely to tear. However, they may take a longer period of time to adjust to as compared to soft contact lenses. They also require a more complex cleaning and disinfection process than soft contact lenses.

Hard contact lenses can be made of the polymer polymethyl methacrylate, which is also known as PMMA, Plexiglas, or Perspex. PMMA is hydrophobic, which helps these lenses repel proteins. These rigid lenses don't use water or silicone to allow for breathability. Instead, fluorine can be added to the polymer, which forms microscopic pores in the material to make a rigid gas permeable lens. Another option is to add methyl methacrylate (MMA) with TRIS to increase the permeability to the lens.

“Hybrid contact lenses” typically have a rigid gas-permeable center attached to an outer “skirt” made of soft contact lens material. The soft, outer part of the lens increases comfort and helps the lens to stay centered on the eye, while the rigid gas permeable center provides clear vision. This design is intended for people who have irregular corneas.

As used herein, “daily wear contact lenses” refers to contact lenses intended for use during the day. They are not designed for overnight wear.

“As used herein, “extended wear contact lenses” refers to contact lenses that can be used overnight from one to about six nights or up to about 30 days. Extended wear contact lenses are usually made of soft plastics that allow more oxygen to pass through to the cornea. Length of continuous wear depends on the contact lens type and tolerance of a patient for overnight wear.

As used herein, “disposable contact lenses” refers to contact lenses that are used once and then thrown away.

As used herein, “replacement contact lenses” refers to contact lenses that may be removed nightly and re-worn for multiple days. Replacement schedules for these contact lenses can vary from about seven to about 30 days.

Typically contact lenses completely cover the cornea and extend by about 2 mm onto the bulbar conjunctiva, with any well-fitting contact lens moving along the conjunctiva with every blink. As used herein “the eye” refers to the cornea and/or conjunctiva unless otherwise indicated.

“Diagnosis” in the context of the present disclosure relates to the determination of a predisposition of the patient to develop symptomatic contact lens wear. A diagnosis may include a comparative assessment to an earlier diagnosis.

“Prognosis” denotes a prediction of whether a patient is likely to experience contact lens discomfort. Prognosis may also include a prediction of response of the patient to treatment.

The term “patient” as used herein refers to a human subject in need of vision correction.

The term “subject” as used herein includes “normal” human subjects that are not in need of vision correction and asymptomatic lens wearers.

The term “sample” as used herein refers to a tear fluid sample collected for the purpose of determining predisposition of a patient to symptomatic contact lens wear. The tear sample is preferably taken from the patient prior to contact lens wear.

The tear sample may be collected using any suitable method that permits measurement of one or more of IL-18, IL-23, and IL-33 in the collected sample. In one example, the tear sample is collected by a microcapillary tube from, for example, the inferior-temporal cul de sac of the patient. In another example, the tear sample is collected using an absorbent strip of material. Suitable materials for collecting tear samples are known in the art (see e.g. Sia et al., Int. J. Opthalmol. Clin. Res. (2016) 3:48). During collection of the tear sample, care is taken to avoid any irritation to the eye to avoid reflex tearing so that only basal tears are collected.

Basal tears are a complex biofluid that bathe the outer surface of the eye. Their composition reflects contributions from multiple sources, including the lacrimal gland (derived as a filtrate from blood), the ocular surface epithelia and the ocular surface vasculature (e.g., conjunctival blood vessels). Levels of pro-inflammatory cytokines, and the time-course of their upregulation, do not necessarily correlate between specific tissues of the eye and the net change in the ocular surface system, as reflected in the basal tear samples (see, e.g., Alven et al., Optom. Vis. Sci. (2021) 98 (11): 1231-1238 and Mohammed et al., Clin. Exp. Ophthalmol. (2020) September; 48 (7): 973-982). Advantageously therefore, pro-inflammatory cytokine levels in the basal tears can be considered to provide an overall (nett) representation of the inflammatory status of the ocular surface, rather than being limited to represent a single contributing tissue/site.

Tears for one or both eyes may be collected. For example, up to about 10 μL/eye may be collected. The skilled person will appreciate that the sample volume needed will depend on the detection assay used (e.g., based on its sensitivity). When tears from both eyes are collected, the collected tears may be pooled to make a single sample. Alternatively, the level of one or more of IL-18, IL-23, and IL-33 may be determined in both of the collected tear samples (i.e., the sample from the left eye, and the sample from the right eye) and optionally, averaged.

The sample may be treated, for example, diluted, concentrated, fractionated, or purified (e.g., partially purified) prior to analysis. The sample may also be frozen prior to analysis.

The term “prior to contact lens wear” as used herein refers to a time point that the sample is taken from a subject. The term is not intended to exclude a subject who has at some point worn contact lenses. Preferably, the subject has not worn contact lenses for a sufficient time for the ocular surface to not be acutely affected by contact lens wear, for example, at least about 24 hours. This sample is sometimes referred to herein as providing a baseline level of one or more of IL-18, IL-23, and IL-33 in the subject.

The term “level” as used herein relates to the concentration (preferably expressed as weight/volume; w/v) of one or more of IL-18, IL-23, and IL-33 in the sample. In some embodiments, the level of one or more of IL-18, IL-23, and IL-33 is the average level of the one or more of IL-18, IL-23, and IL-33 in tear samples collected from the left and right eyes.

Determining the level of one or more of IL-18, IL-23, and IL-33 in a sample includes both quantitative and/or qualitative determinations. Thus, in one example, the concentration of one or more of one or more of IL-18, IL-23, and IL-33 in the sample, typically in units of pg/ml, may be measured. In another example, the amount of one or more of IL-18, IL-23, and IL-33 in the sample may be determined to be higher or lower than that of a reference level. In yet another example, the amount of the one or more of IL-18, IL-23, and IL-33 in the sample may be determined to be higher or lower than that of a cut-off value predetermined to be associated with a predisposition to symptomatic contact lens wear.

Any suitable “assay” or “diagnostic assay” can used to determine the level of one or more of IL-18, IL-23, and IL-33 in the sample. Such an assay may be based on the binding of an analyte (i.e., IL-18, IL-23, or IL-33) to be detected to one or more capture probes with a certain affinity. Concerning the interaction between capture molecules and target molecules or molecules of interest, the affinity constant is preferably greater than 108 M.

In the context of the present disclosure, “capture molecules” are molecules which may be used to bind target molecules or molecules of interest, i.e., analytes (i.e., in the context of the present disclosure, IL-18, IL-23, or IL-33) in a sample. Capture molecules must thus be shaped adequately, both spatially and in terms of surface features, such as surface charge, hydrophobicity, hydrophilicity, presence or absence of Lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present disclosure, capture molecules may for instance be selected from the group comprising a nucleic acid molecule, a carbohydrate molecule, a PNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments.

The preferred detection methods comprise immunoassays in various formats such as for instance radioimmunoassay (RIA), chemiluminescence- and fluorescence-immunoassays, Enzyme-linked immunoassays (ELISA), bead arrays, protein microarray assays, and rapid test formats such as for instance immunochromatographic strip tests. Other detection methods include proteomic/mass-spectrometry techniques known to those skilled in the art and may be used to quantify levels of tear IL-18, IL-23, or IL-33.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschis et al., Curr, Opin, Chem, Biol. (2006) 10(1):4-10).

In one example, the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

The labelling system may comprise rare earth cryptates or rare earth chelates in combination with fluorescence dye or chemiluminescence dye, in particular a dye of the cyanine type.

In the context of the present disclosure, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, CY7, Xanthen, 6-Carboxy-2′,4′,7\4,7-hexachlorofluorescein (HEX), TET, 6-Carboxy-4′,5′-dichloro-2′,7′-dimethoxyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-Carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present disclosure, chemiluminescence based assays comprise the use of dyes, based on the physical principles described for chemiluminescent materials in for example, Kirk-Othmer, Encyclopedia of chemical technology, 4th ed., executive editor, J. I. Kroschwitz; editor, M. Howe-Grant, John Wiley & Sons, 1993, vol. 15, p. 518-562 including citations on pages 551-562. Preferred chemiluminescent dyes are acridiniumesters.

In one embodiment, the assay is a cytometric bead array—a flow cytometry application that allows users to quantify multiple proteins simultaneously. The system uses the broad dynamic range of fluorescence detection offered by flow cytometry and antibody-coated beads to efficiently capture analytes. Each bead in the array has a unique fluorescence intensity so that beads can be mixed and run simultaneously in a single tube. This method significantly reduces sample requirements and time to results in comparison with traditional ELISA and Western blot techniques. In one embodiment, the array is used to simultaneously measure the level of two or all of IL-18, IL-23, or IL-33 in the sample.

The term “correlating” or “correlated” as used herein refers to comparing the level of the one or more of IL-18, IL-23, and IL-33 in the sample to its amount in asymptomatic contact lens wearers, or subjects known to have, or known to be at risk of contact lens discomfort, or subjects known to respond or not respond to a given treatment.

The level of one or more of IL-18, IL-23, and IL-33 in a sample can be compared to a level known to be indicative of an abnormal state (i.e., symptomatic contact lens wear). In some embodiments, the level has been correlated with a diagnosis; that is, the skilled person can use the level to determine whether the patient is predisposed to symptomatic contact lens wear and respond accordingly. Alternatively, the level can be compared to a level indicative of a normal state. In some embodiments, the level has been correlated with the absence of symptomatic contact lens wear. In other embodiments, the level can be compared to a level of the one or more of IL-18, IL-23, and IL-33 in a sample taken from the patient at an earlier timepoint to, for example, monitor treatment efficacy. In some embodiments, the level can be compared to a baseline level of the one or more of IL-18, IL-23, and IL-33 in a sample taken before initiation of treatment or following a round of treatment. In other embodiments, the level has been correlated with a response or non-response to a given treatment.

The sensitivity and specificity of a diagnostic and/or prognostic test depends on more than just the analytical “quality” of the test, they also depend on the definition of what constitutes an abnormal result. Receiver Operating Characteristic curves (ROC curves) can be calculated by plotting the value of a variable versus its relative frequency in “normal” (i.e., apparently healthy) or asymptomatic contact lens wearers and “condition” populations (i.e., having symptomatic contact lens wear). For any particular marker, a distribution of IL-18, IL-23, or IL-33 levels for subjects with and without symptomatic contact lens wear will likely overlap. Under such conditions, a test does not absolutely distinguish normal from condition with 100% accuracy, and the area of overlap indicates where the test cannot distinguish normal from condition. A threshold is selected, above which (or below which, depending on how a marker changes with the condition) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “condition” samples might be ranked according to degree (e.g., 1=low, 2=normal, and 3=high). This ranking can be correlated to results in the “normal” population or to asymptomatic contact lens wearers, and a ROC curve created. These methods are well known in the art. Preferably, ROC curves result in an AUC of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The threshold level may be a weighted threshold level where the level of one or more of IL-18, IL-23, or IL-33 has been adjusted using a scaling factor (including fraction and integer multiplications) to improve the specificity or sensitivity or both the specificity or sensitivity of the diagnostic and/or prognostic test. For example, the level of IL-23 may be scaled up by a factor of about 2 and IL-33 scaled down by a factor of 0.5 when used in combination to diagnose/prognose symptomatic contact lens wear in a patient.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the discriminative capacity of the test.

In certain embodiments, the level of one or more of IL-18, IL-23, and IL-33 as a marker exhibits at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%), more preferably at least about 80%, even more preferably at least about 85%, still more preferably at least about 90%, and most preferably at least about 95%. The term “about” in this context refers to +/−5% of a given measurement.

Preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

In some examples, the diagnostic threshold level of predisposition to symptomatic contact lens wear for tear IL-18 is about >128 pg/ml as determined by multiplex cytometric bead array (e.g., LEGENDplex Human Inflammation Panel 1, BioLegend, #740809). This equates to about 75% (95% CI: 51% to 90%) sensitivity and about 73% specificity (95% CI: 43% to 91%).

In some examples, the diagnostic threshold level of predisposition to symptomatic contact lens wear for tear IL-23 is about >33 pg/ml as determined by multiplex cytometric bead array (e.g., LEGENDplex Human Inflammation Panel 1, BioLegend, #740809). This equates to about 80% (95% CI: 55% to 93%) sensitivity and about 64% specificity (95% CI: 36% to 85%).

In other examples, the diagnostic threshold level of predisposition to symptomatic contact lens wear for tear IL-23 is about >29 pg/ml as determined by multiplex cytometric bead array (e.g., LEGENDplex Human Inflammation Panel 1, BioLegend, #740809). This equates to about 87% (95% CI: 62% to 93%) sensitivity and about 55% specificity (95% CI: 28% to 88%).

In other examples, the diagnostic threshold level of predisposition to symptomatic contact lens wear for tear IL-23 is about >46 pg/ml as determined by multiplex cytometric bead array (e.g., LEGENDplex Human Inflammation Panel 1, BioLegend, #740809). This equates to about 67% (95 CI: 42% to 85%) sensitivity and about 91% specificity (95% CI: 63% to 100%).

In some examples, the diagnostic threshold level of predisposition to symptomatic contact lens wear for tear IL-33 is about >170 pg/ml as determined by multiplex cytometric bead array (e.g., LEGENDplex Human Inflammation Panel 1, BioLegend, #740809). This equates to about 67% (95% CI: 42% to 85%) sensitivity and about 82% specificity (95% CI: 52% to 97%).

In other examples, the diagnostic threshold level of predisposition to symptomatic contact lens wear for tear IL-33 is about >125 pg/ml as determined by multiplex cytometric bead array (e.g., LEGENDplex Human Inflammation Panel 1, BioLegend, #740809). This equates to about 73% (95% CI: 49% to 89%) sensitivity and about 55% specificity (95% CI: 28% to 79%).

In other examples, the diagnostic threshold level of predisposition to symptomatic contact lens wear for tear IL-33 is about >368 pg/ml as determined by multiplex cytometric bead array (e.g., LEGENDplex Human Inflammation Panel 1, BioLegend, #740809). This equates to about 40% (95 CI: 20% to 64%) sensitivity and about 91% specificity (95% CI: 63% to 100%).

In some examples, the diagnostic threshold level of predisposition to symptomatic contact lens wear is tear IL-23 about >50 pg/ml OR tear IL-33 about >200 pg/ml as determined by multiplex cytometric bead array (e.g., LEGENDplex Human Inflammation Panel 1, BioLegend, #740809). This equates to about 73% sensitivity and about 100% specificity.

In other examples, the diagnostic threshold level of predisposition to symptomatic contact lens wear is tear IL-23 about >50 pg/ml OR tear IL-33 about >370 pg/ml as determined by multiplex cytometric bead array (e.g., LEGENDplex Human Inflammation Panel 1, BioLegend, #740809). This equates to about 60% sensitivity and about 100% specificity.

In other examples, the diagnostic threshold level of predisposition to symptomatic contact lens wear is tear IL-23 about >50 pg/ml OR tear IL-33 about >370 pg/ml OR tear IL-18 about >128 pg/ml as determined by multiplex cytometric bead array (e.g., LEGENDplex Human Inflammation Panel 1, BioLegend, #740809). This equates to about 80% sensitivity and 70% specificity.

In other examples, the diagnostic threshold level of predisposition to symptomatic contact lens wear is tear ((IL-23×2)+(IL-33×0.5))>160 pg/ml as determined by multiplex cytometric bead array (e.g., LEGENDplex Human Inflammation Panel 1, BioLegend, #740809). This equates to about 73% sensitivity and 82% specificity.

In some embodiments, the level of the one or more of IL-18, IL-23, and IL-33 in the sample is combined with one or more clinical parameters selected from the group consisting of age, gender, suboptimal tear quality and/or quantity, clinical history of dry eye disease or allergic eye disease, use of medications or presence of systemic conditions known to affect ocular surface health, use of contact lenses in suboptimal environment (e.g., low humidity, exposure to high temperature or wind), and baseline eye discomfort or pain, or elevated tear IL-17A levels measured during the day whilst wearing a contact lens.

The term “combined” or variations such as “combination” or “combining” is defined as a possible selection of a certain number of parameters and the arrangement of these parameters into specified groups using a mathematical algorithm (e.g., deviation or ratio). For example, a ratio can be calculated between the level of a biomarker (e.g., IL-18, IL-23, or IL-33) in a sample taken from a patient and the level of the same biomarker in a sample taken from the patient at an earlier time i.e., a baseline level (e.g., taken before the start of treatment). Moreover, a deviation can be calculated between the level of a biomarker in a sample taken from a patient and the level of the same biomarker in a sample taken from the patient at an earlier time point. It is also encompassed herein, that a ratio between different biomarkers can be calculated. For example, the ratio can be calculated between biomarker levels measured in samples taken from the patient at the same time point or at different time points.

In some embodiments, the level of the at least one biomarker can be combined as continuous or categorical variable.

The term “score” in the context of the present disclosure refers to a rating, expressed numerically, based on the specific achievement or the degree to which certain qualities (e.g., the level of one or more of IL-18, IL-23, and IL-33) are present in the sample.

The methods described herein advantageously enable the eye-care professional to provide better patient care and management, which may lead to higher patient satisfaction and retention. The eye-care professional may provide different aftercare scheduling or advice to a patient who is determined to be predisposed to symptomatic contact lens wear compared to a patient who is determined to be predisposed to asymptomatic contact lens wear. For example, a patient that is determined to be predisposed to symptomatic contact lens wear may be prescribed a more comfortable, premium lens and/or an eye treatment such as wetting eye drops that promote ocular comfort, anti-inflammatory agents such as corticosteroids, or other treatments (including those for dry eye disease for example) that may attenuate contact lens discomfort including immunomodulatory agents, therapeutics for neuropathy or ocular pain or discomfort, tear composition modifying agents, viscoelastic agents, tear retention agents (e.g., punctal plugs), tear stimulatory agents (e.g., neurostimulation or secretagogue), ocular anti-allergy agents. Other treatments also include an environmental modification (e.g., use of a humidifier, avoidance of air drafts, etc.) or use of a different contact lens disinfection system.

The term “treatment stratification” in the context of the present disclosure refers to the choice and/or adjustment of a therapeutic treatment of the patient.

The term “risk stratification” in the context of the present disclosure refers to a probability of specified outcomes for a patient.

The skilled person will appreciate that the diagnostic or prognostic levels given herein have been calculated based on a specific assay. And that such absolute levels may change depending on how IL-18, IL-23, or IL-33 levels are detected in the tear fluid sample. It would be routine in the art for the skilled person to calculate new diagnostic levels of tear IL-18, IL-23, or IL-33 detected using a different assay, now that one or more of IL-18, IL-23, and IL-33 has been shown to be a diagnostic biomarker of contact lens discomfort.

The following Examples illustrate certain aspects and advantages of the present disclosure, which should be understood not to be limited thereby.

### EXAMPLES

### Methods

This was a prospective, longitudinal clinical study undertaken at the University of Melbourne, Victoria, Australia. Eligible study participants were adult soft contact lens wearers, currently wearing silicone-hydrogel contact lenses. The key study participant eligibility criteria at enrolment were:
Silicone hydrogel contact lens wearer for 6 months or more;
Wearing presenting type of contact lens, and using the presenting contact lens disinfection solution (if relevant) for at least one month;
Full-time soft contact lens wearer, defined as routinely wearing contact lenses at least four times per week;
Best-corrected visual acuity (BCVA) of at least 20/40 in each eye;
No change to medications that might affect tear film status in the past three months;
No other ocular significant ocular conditions other than contact lens discomfort.

### Overview of Participant Visit Schedule:

Participants attended a total of four study visits:
Day 1—Visit 1: baseline—no contact lens (CL) wear.
Day 7±3: CL-wearing day, with 3 visits over this day:
   Visit 2: Morning, prior to CL wear (and with no CL wear for at least 24 hours prior): habitual CLs applied (for wear throughout the day) at the end of this visit.
   Visit 3: Lunchtime (~3-5 hours CL wearing time)
   Visit 4: End-of day (~7-10 hours CL wearing time)
      A full schedule of study procedures is provided in Table 1.

TABLE 1
Schedule of study procedures

| | VISIT 1 | VISIT 2 | VISIT 3 | VISIT 4 |
|---|---|---|---|---|
| | Day 1 | Day 7 ± 3 | Day 7 ± 3 | Day 7 ± 3 |
| | Baseline - | Baseline | CL wear | CL wear |
| | no CL | on day of | (4-5 | (8-10 |
| Procedure | wear) | CL wear# | hours) | hours) |
| Written informed consent | ✓ | | | |
| Demographics, medical and ophthalmic history | ✓ | | | |
| Concomitant medication query | ✓ | ✓ | | |
| Dry eye questionnaire (OSDI) | ✓ | | | |
| CLD questionnaire: CLDEQ-8 | ✓ | | | |
| Visual Analogue Scale (VAS) for ocular comfort and | ✓ | ✓ | ✓ | ✓ |
| Ocular testing procedures | | | | |
| Best-corrected visual acuity (BCVA) | ✓ | | | |
| Tear osmolarity (including I-SOD¹ in both eyes) | ✓ | ✓ | | ✓* |
| Non-invasive tear break-up time (NITBUT) | ✓ | ✓ | ✓* | ✓* |
| Basal tear collection (for cytokine analyses)² | ✓ | ✓ | ✓* | ✓* |
| Non-contact corneal aesthesiometry (NCCA)³ | | ✓ | | ✓\hat{ } |
| Slit lamp examination⁴ of the ocular surface: | ✓ | | | |
| Bulbar redness assessment | ✓ | | | |
| Limbal hyperaemia | ✓ | | | |
| Eyelid evaluation (including meibomian glands) | ✓ | | | |
| Tear break up time with fluorescein (TBUT) | ✓ | | | |
| Corneal sodium fluorescein staining (Oxford) | ✓ | | | ✓\hat{ } |
| Conjunctival lissamine green staining (Oxford) | ✓ | | | ✓\hat{ } |
| Lid wiper epitheliopathy assessment | ✓ | | | ✓\hat{ } |
| Schirmer test with anaesthesia | ✓ | | | |
| Infrared meibography | ✓ | | | |
| Eligibility determination | ✓ | | | |

¹ISOD: ‘Inferior-Superior Osmotic Difference’, I-SOD, defined as the absolute osmolarity difference between these menisci.
²Basal tear collection was performed prior to CL wear at Visits 1&2, and with the CLs in situ at Visits 3&4.
³NCCA was performed in the central cornea using ambient and cooled (5 °C below ambient) air stimuli
⁴Slit lamp assessment involve using standardised grading scales (Efron) for bulbar redness, limbal hyperaemia and eyelids, including meibomian gland evaluation), tear break up time (TBUT), corneal sodium fluorescein staining (Oxford scale) and conjunctival lissamine green staining (Oxford scale).
*Performed over the CL surface (without disturbing the CL on eye)
\hat{ }Performed post-CL removal.
^{~}for females of childbearing potential only.
#CLs inserted at the end of this visit (Visit 2).

### Tear Collection:

Basal tear collection was performed prior to CL wear at Visits 1 and 2, and with the CLs in situ at Visits 3 and 4. Non-stimulated (basal) tear samples (up to 10 μl/eye) were collected from the inferior-temporal cul de sac of participants' eyes using a glass microcapillary tube (Drummond Scientific 20 μl MicroCap), using our established protocols. Tear flow rate was monitored to exclude dilution effects caused by reflex tearing. Only samples with a flow rate of 1 to 3 μl/min were used. Following collection, samples were stored at −80 °C until required for the analyses.

### Tear Analyses:

Tear samples were quantified for multiple analytes (Table 2), comprising: i) CGRP using an enzyme immunoassay (Human CGRP ELISA, Phoenix Pharmaceuticals, #EK-015-02), according to the manufacturer's instructions; and ii) pro-inflammatory cytokines using a multiplex cytometric bead array (LEGENDplex Human Inflammation Panel 1, BioLegend, #740809) with samples acquired on a Becton Dickinson FACSCanto II flow cytometer (Franklin Lakes, NJ, US), and data analysed using BioLegend's LEGENDplex data analysis software.

Wherever possible (as determined by the amount of tear sample available for analysis), data from the right and left eye of an individual participant were averaged to yield a single value for that person. Otherwise, data from a single eye were used.

TABLE 2
Summary of tear analytes considered in this report

| | Type of | |
|---|---|---|
| Analyte | mediator | Role/Activity |
| CGRP | Neuropeptide | Released from corneal nerves, and |
| | | mediates pain |
| | | Trophic role between corneal nerves and |
| | | epithelial cells |
| IL-23 | Cytokine | Inflammation, Th17 responses |
| | (from the | Produced by dendritic cells, which causes |
| | IL-12 | development of Th17 cells (i.e., IL-23 is the |
| | family) | inducing factor of Th17 cells, and IL-17A is |
| | | the effect factor of Th17 cells) |
| IL-33 | Cytokine | Inflammation, Th2 responses |
| | (member of | Expressed by epithelial cells, fibroblasts, |
| | the IL-1 | keratinocytes, dendritic cells and activated |
| | family) | macrophages |
| | | Implicated in asthma and allergic |
| | | conjunctivitis |
| IL-18 | Cytokine | Inflammation, Th1 responses and Th2 |
| | (member of | responses |
| | the IL-1 | Implicated in several autoimmune diseases |
| | family) | and psoriasis |
| IL-8 / | Chemokine | Chemoattractant for neutrophils |
| CXCL8 | | |

Statistical analyses: Data were analysed using GraphPad Prism 7 software package (GraphPad Software, San Diego, USA). Descriptive statistics in the Figures are summarised as mean±SEM for parametric data, unless otherwise stated. A two-way, repeated measures ANOVA was used to measure tear analyte levels, with a within-participant variable of timepoint (visit number) and between-participant variable of study group (asymptomatic of symptomatic CL wearer). Multiple comparisons testing was performed using the Fisher's LSD test. An alpha value of 0.05 was adopted for statistical significance.

### Results

Data are presented for 11 asymptomatic soft CL wearers and 16 symptomatic soft CL wearers. Baseline (Visit 1) demographic and key clinical characteristics of the study participants are summarized in Table 3.

TABLE 3
Summary of baseline (Visit 1) participant demographics
and key clinical characteristics

| | Asymptomatic CL | Symptomatic CL |
|---|---|---|
| Parameter | wearers (n = 11) | wearers (n = 16) |
| Age (years) | 36.3 ± 13.8 | 34.2 ± 9.6 |
| | [range: 21 to 62] | [range: 18 to 59] |
| Sex (male/female) | 4/7 | 3/13 |
| CL replacement | 2/5/4 | 3/7/6 |
| schedule | | |
| (Daily/fortnightly/monthly) | | |
| CLDEQ-8 score (CL | 4.5 ± 3.0 | 17.5 ± 4.3 |
| discomfort symptom | | |
| score) (/37) | | |
| Ocular Surface Disease | 3.5 ± 4.1 | 11.0 ± 8.1 |
| Index (OSDI) dry eye | | |
| score, when not wearing | | |
| CLs (/100) | | |
| Tear osmolarity (higher | 303.8 ± 6.9 | 304.7 ± 9.8 |
| eye), mOsm/L | | |
| Non-invasive tear break- | 9.5 ± 3.5 | 12.2 ± 3.7 |
| up time (NITBUT), secs | | |
| Corneal fluorescein | 1.2 ± 0.9 | 1.0 ± 0.9 |
| staining grade (/5) | | |
| Schirmer test score | 12.9 ± 7.1 | 8.5 ± 8.3 |
| (mm/5 min) | | |

Data shown as mean ± standard deviation (SD).

The CLDEQ-8 score was significantly higher in the symptomatic CL wearers, compared to asymptomatic CL wearers; all other key demographic and clinical features were similar (Table 3).

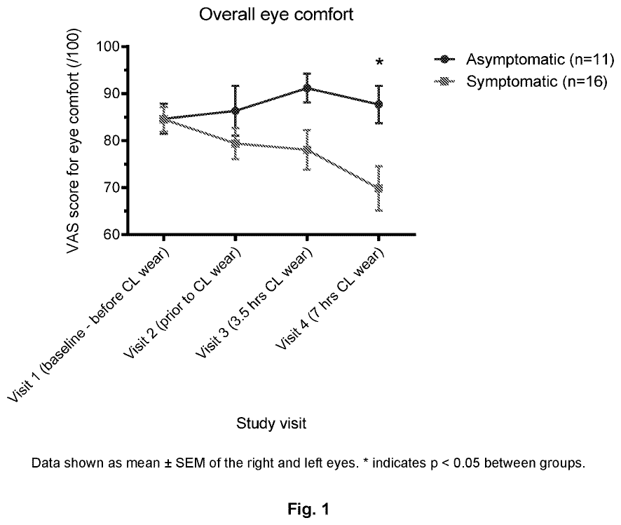

The ocular comfort characteristics are shown in FIG. 1. Ocular comfort was significantly lower (p<0.05) in the symptomatic group relative to asymptomatic CL wearers at Visit 4.

### Tear Analytes:

### CGRP

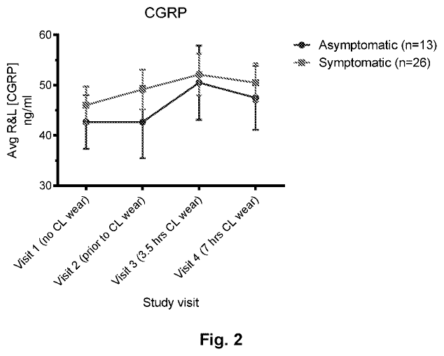

Tear levels of CGRP were similar at Visits 1 and 2, in the absence of CL wear. As shown in FIG. 2, after 3.5 hours of CL wear (Visit 3), there was a general trend towards higher tear CGRP levels, although this was not statistically significant within or between groups.

### IL-23 and IL-33

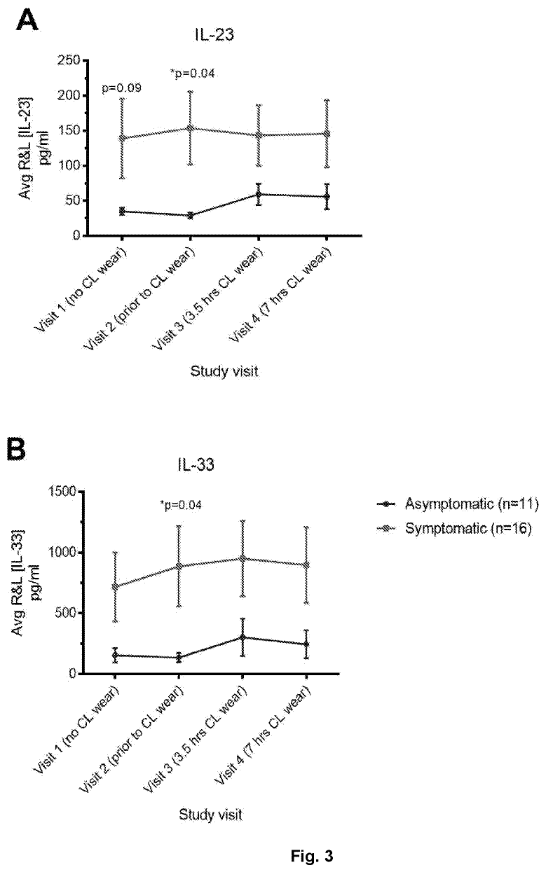

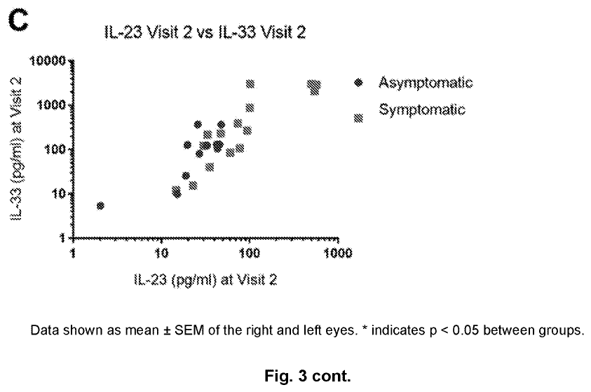

Findings for tear IL-23 (FIG. 3A) and IL-33 (FIG. 3B) levels are shown together, as their pattern across visits was similar. Tear levels of both pro-inflammatory cytokines were significantly higher at Visit 2 (prior to CL wear) in symptomatic CL wearers. There was a strong linear relationship (R²=0.69, p<0.0001) between tear IL-23 and IL-33 levels at Visit 2 (FIG. 3C).

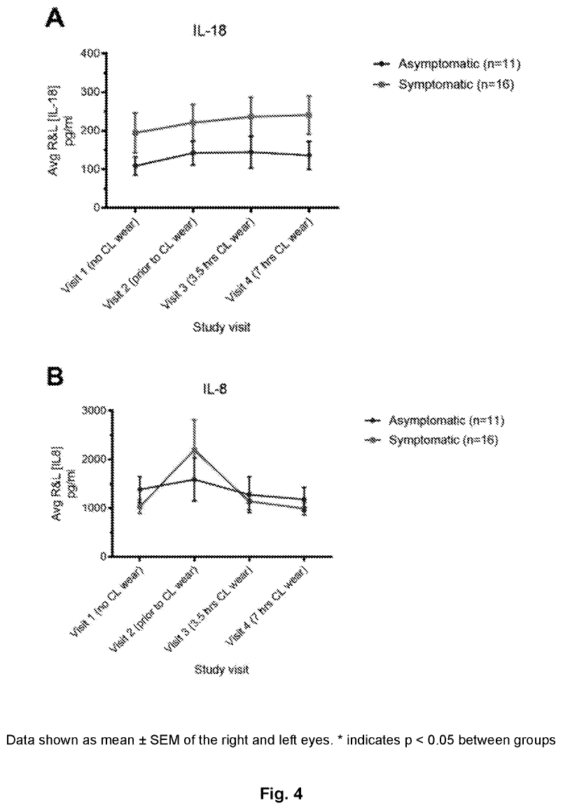

There was a trend towards symptomatic CL wearers having higher overall levels of tear IL-18 (FIG. 4A).

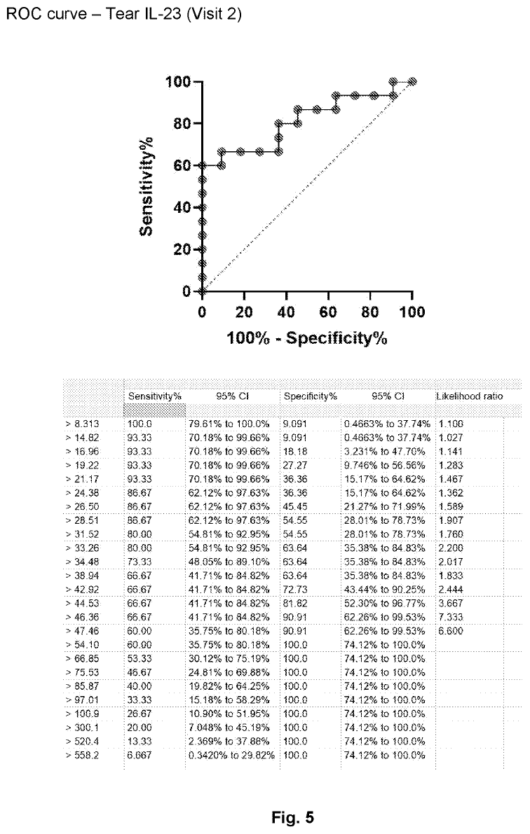

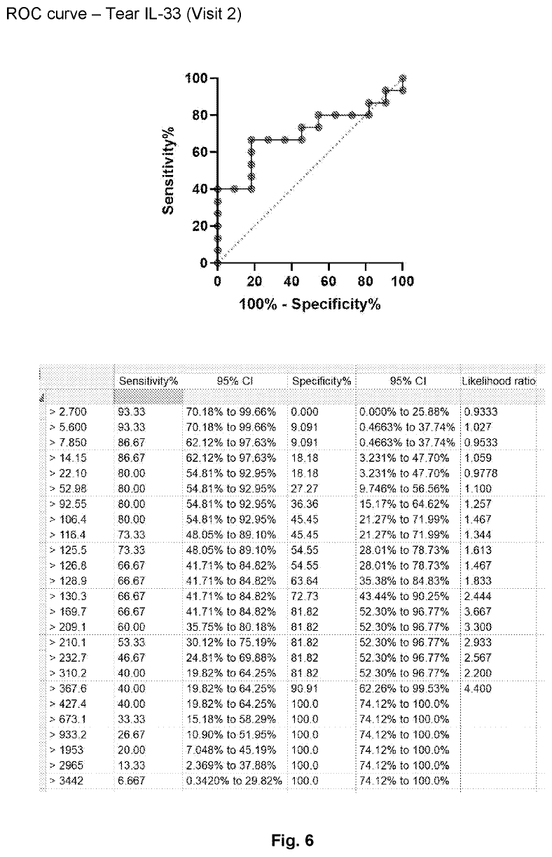

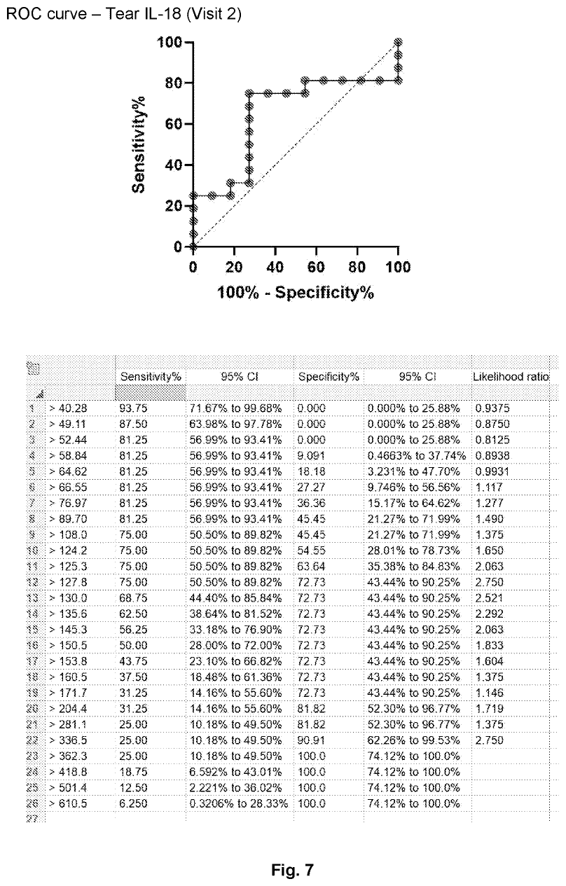

The outcome of the receiver operator characteristic (ROC) curve analyses, to investigate the prognostic utility to identify individuals predisposed to CL discomfort, are shown for tear IL-23 (FIG. 5), tear IL-33 (FIG. 6) and tear IL-18 (FIG. 7) at Visit 2. These plots show that for:
Tear IL-23, the Area Under the Curve (AUC) is 0.81 (standard error: 0.085; 95% CI: 0.65 to 0.98 (p=0.0075)). The associated sensitivity/specificity table (FIG. 5) shows that the diagnostic threshold level of predisposition to symptomatic CL wear for tear IL-23 is about: >33 pg/ml for about 80% (95% CI: 55% to 93%) sensitivity and about 64% (95% CI: 36% to 85%) specificity; >29 pg/ml for about 87% (95% CI: 62% to 93%) sensitivity and 55% (95% CI: 28% to 88%) specificity; >46 pg/ml for about 67% (95 CI: 42% to 85%) sensitivity and about 91% (95% CI: 63% to 100%) specificity.
Tear IL-33, the AUC is 0.70 (standard error: 0.10; 95% CI: 0.50 to 0.91 (p=0.08)). The associated sensitivity/specificity table (FIG. 6) shows that the diagnostic threshold level of predisposition to symptomatic CL wear for tear IL-33 is about: >170 pg/ml for about 67% (95% CI: 42% to 85%) sensitivity and about 82% (95% CI: 52% to 97%) specificity; >125 pg/ml for about 73% (95% CI: 49% to 89%) sensitivity and about 55% (95% CI: 28% to 79%) specificity; >368 pg/ml for about 40% (95 CI: 20% to 64%) sensitivity and about 91% (95% CI: 63% to 100%) specificity.
Tear IL-18, the AUC is 0.65 (standard error: 0.11; 95% CI: 0.43 to 0.87 (p=0.20)). The associated sensitivity/specificity table (FIG. 7) shows that the diagnostic threshold of predisposition to symptomatic CL wear for tear IL-18 is about >128 pg/ml for about 75% (95% CI: 51% to 90%) sensitivity and about 73% (95% CI: 43% to 91%) specificity.

In addition, scaling factors (ranging from fractions to integer multiplications) were iteratively applied to one or more of each cytokine (i.e., to the levels of tear IL-23, IL-33 and IL-18 at Visit 2), in combination with each other, to consider whether the capacity to determine predisposition to symptomatic CL wear was enhanced using a combination of tear cytokine levels. These analyses identified that a diagnostic threshold of predisposition to symptomatic CL wear could be derived using: tear ((IL-23×2)+(IL-33×0.5))>160 pg/ml, to give about 73% sensitivity and about 82% specificity.

### DISCUSSION

The present findings support a role for immune-mediated inflammatory responses in the pathogenesis of contact lens discomfort. These data provide new insight into the dynamics of tear pro-inflammatory cytokine levels, and identify three new potential biomarker candidates, alone and/or combination, that were constitutively elevated in wearers who were predisposed to CL discomfort.

Relative to asymptomatic, soft disposable silicone-hydrogel CL wearers, symptomatic wearers had significantly higher basal (pre-CL wear) levels of both tear IL-23 and IL-33; levels of these cytokines were unchanged throughout the wearing day. There was a moderately strong correlation between baseline tear levels of IL-23 and IL-33 in individual study participants. There was also a strong trend towards symptomatic wearers having overall higher levels of tear IL-18 (FIG. 4).

The claims defining the invention are as follow:

1. A method of determining a predisposition to symptomatic contact lens wear in a patient said method comprising:
a) determining the concentration of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33,
wherein a higher level of the one or more of IL-18, IL-23, and IL-33 in the tear sample compared to the reference level is indicative of a predisposition of the patient to symptomatic contact lens wear.

2. The method of claim 1, wherein the level of the one or more of IL-18, IL-23, and IL-33 in the tear sample is determined prior to contact lens wear.

3. The method of claim 1, wherein the reference level of the one or more of IL-18, IL-23, and IL-33 is a level of the one or more of IL-18, IL-23, and IL-33 in a sample taken prior to contact lens wear from a subject who is asymptomatic to contact lens wear.

4. The method of any one of claims 1 to 3, wherein the reference level of the one or more of IL-18, IL-23, and IL-33 is a threshold level.

5. The method of claim 4, wherein the threshold level is a cut-off value.

6. The method of any one of claims 1 to 5, wherein the method further comprises obtaining the tear sample from the patient.

7. The method of any one of claims 1 to 6, wherein the level of the one or more of one of IL-18, IL-23, and IL-33 is determined using an immunoassay such as a multiplex cytometric bead array.

8. The method of claim 7, wherein the threshold level for:
i) tear IL-18 is about >128 pg/ml; or
ii) tear IL-23 is between about >29 to about >46 pg/ml; or
iii) tear IL-33 is between about >170 to >368 pg/ml.

9. The method of claim 7, wherein the threshold level for:
i) tear IL-23 is about >50 pg/ml OR tear IL-33 is about >200 pg/ml; or
ii) tear IL-23 about >50 pg/ml OR tear IL-33 is about >370 pg/ml; or
iii) tear IL-23 is about >50 pg/ml OR tear IL-33 is about >370 pg/ml OR tear IL-18 is about >128 pg/ml.

10. The method of any one of claims 1 to 7, wherein the method further comprising applying a scaling factor to the level of one or more of one of IL-18, IL-23, and IL-33.

11. The method of claim 10, wherein the threshold level is a weighted threshold level given by the formula:
i) (tear IL-23×scaling factor A)+(tear IL-33×scaling factor B); or
ii) (tear IL-23×scaling factor A)+(tear IL-18×scaling factor C); or
iii) (tear IL-33×scaling factor B)+(tear IL-18×scaling factor C); or
iv) (tear IL-23×scaling factor A)+(tear IL-33×scaling factor B)+(tear IL-18×scaling factor C).

12. The method of claim 11, wherein the weighted threshold level is:
(tear IL-23×about 2)+(tear IL-33×about 0.5))>about 160 pg/ml.

13. The method of any one of claims 1 to 12, further comprising selecting a treatment or modifying a treatment, based on the diagnosis of a predisposition of the patient to symptomatic contact lens wear.

14. The method of claim 13, wherein the treatment is for preventing, inhibiting, or reducing discomfort associated with contact lens wear and is selected from administration of a therapeutic agent for neuropathy or ocular pain or discomfort, an anti-inflammatory agent, a wetting agent, an immunomodulatory agent, a tear composition modifying agent, a viscoelastic agent, a different contact lens, a different contact lens disinfection system, a tear retention agent (e.g., punctal plugs), a tear stimulatory agent (e.g., neurostimulation or secretagogue), a dry eye treatment, an ocular anti-allergy agent, an environmental modification (e.g., use of a humidifier, avoidance of air drafts, etc.) and combinations thereof.

15. The method of claim 13 or 14, wherein the treatment is given prophylactically to prevent or reduce discomfort associated with contact lens wear.

16. The method of any one of claims 13 to 15, wherein the treatment is given during contact lens wear to inhibit or reduce severity of discomfort associated with contact lens wear.

17. The method of claim 13 or 14 wherein the treatment is for preventing or reducing discomfort associated with contact lens wear and comprises prescribing the patient spectacle lenses for vision correction.

18. The method of any one of claims 1 to 17, wherein the patient does not have Sjogren's syndrome or dry eye disease.

19. A method for selecting a treatment for a patient in need of vision correction, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of L-18, IL-23, and IL-33; and
c) selecting a treatment for preventing, inhibiting, or reducing discomfort associated with contact lens wear when the level of the one or more of IL-18, IL-23, and IL-33 is higher in the tear sample compared to the reference level.

20. A method for treating a patient in need of vision correction, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33; and
c) prescribing the patient a treatment for preventing, inhibiting, or reducing discomfort associated with contact lens wear when the level of the one or more of IL-18, IL-23, and IL-33 is higher in the tear sample compared to the reference level.

21. A method of monitoring treatment efficacy of a patient in need of vision correction and having a predisposition to symptomatic contact lens, or a patient in need of vision correction having symptomatic contact lens wear, said method comprising:
a) determining the level of one or more of interleukin-18 (IL-18), interleukin-23 (IL-23), and interleukin-33 (IL-33) in a tear sample from the patient; and
b) comparing the level of the one or more of IL-18, IL-23, and IL-33 in the tear sample to a reference level of the one or more of IL-18, IL-23, and IL-33; and
c) optionally, continuing treatment when the subject is determined to be a responder or has a decreased level of the one or more of IL-18, IL-23, and IL-33 compared to the reference level; or switching the treatment when the subject is determined to be a non-responder, wherein switching the treatment comprises increasing a dose of the treatment administered, adding one or more of agents to the treatment regimen, or discontinuing the treatment and initiating a different treatment.

22. Use of one or more of interleukin-18 (IL-18), interleukin-23 (IL-23), and interleukin-33 (IL-33) as a tear biomarker for predisposition to symptomatic contact lens wear.
